(19) United States
(12) Patent Application Publication
(10) Pub. No.: US 2026/0306182 A1
(43) Pub. Date: Oct. 8, 2026

(54) SURGICAL TOOL SELECTION AND OPERATION

(71) Applicant: Worcester Polytechnic Institute, Worcester, MA (US)

(72) Inventors: Giovanni Pittiglio, Worcester, MA (US); Aabha Tamhankar, Worcester, MA (US)

(21) Appl. No.: 19/638,563

(22) Filed: Apr. 3, 2026

Related U.S. Application Data
(60) Provisional application No. 63/782,851, filed on Apr. 3, 2025.

(51) Int. Cl. A61B 34/10

(52) U.S. Cl. CPC A61B 34/10; A61B 2034/104; A61B 2034/105; A61B 2034/107

(57) ABSTRACT

Endovascular procedures such as neuroendovascular treatment of stroke generally involve insertion of a surgical catheter and guidewire through the vascular structures (blood vessels) of a patient to access regions of compromise. Both the guidewire and the catheter may be shaped to correspond to a vascular path defined by a natural curvature of the vascular structures as they traverse the patient anatomy. Bends in the guidewire, defined by a series of angular deformations at predetermined lengths of the guidewire, facilitate traversal of the vascular path from coordinated insertions and rotation of the guidewire to advance to the surgical region. An imaging medium typically allows concurrent views of the guidewire traversal within the vascular structures. Selection of an optimal guidewire results from comparison with the database or model generated from previous procedures.

### RELATED APPLICATIONS

This patent application claims the benefit under 35 U.S.C. § 119(e) of U.S. Provisional Patent App. No. 63/782,851, filed Apr. 3, 2025, entitled “SURGICAL TOOL SELECTION AND OPERATION,” incorporated herein by reference in entirety.

### BACKGROUND

Endovascular procedures allow surgical treatment using specialized guidewires and catheters inserted into a blood vessel and traversing the blood vessel to a surgical treatment site. Neuroendovascular access often relies on passive microwires that are manually shaped by physical deformation or bending, and then used to track a microcatheter to the target. Neuroendovascular surgeons determine the shape and mechanical properties of the wire and catheters by examining the patient's pre-operative images and using their experience to identify anatomy-specific shapes of the wire that would facilitate reaching the target. This procedure is particularly complex and time consuming in convoluted anatomical structures and is heavily dependent on the surgeon's level of expertise.

### SUMMARY

Endovascular procedures such as neuroendovascular treatment of stroke generally involve insertion of a surgical catheter and guidewire through the vascular structures (blood vessels) of a patient to access regions of compromise. Both the guidewire and the catheter may be shaped to correspond to a vascular path defined by a natural curvature of the vascular structures as they traverse the patient anatomy. Bends in the guidewire, defined by a series of angular deformations at predetermined lengths of the guidewire, facilitate traversal of the vascular path from coordinated insertions and rotation of the guidewire to advance to the surgical region. An imaging medium typically allows concurrent views of the guidewire traversal within the vascular structures. Selection of an optimal guidewire based on the procedure and anatomy of the patient through automated comparison and classification using a database or model generated from previous procedures facilitates and expedites selection of the surgical guidewire and catheter, mitigating a need for iterative insertions and physical resistance of the vascular path.

Configurations herein are based. In part, on the observation that catheter-delivered guidewires are often employed for surgical procedures such as brain/neurological and cardiac procedures to address conditions such as a thrombectomy, embolization in malformations (e.g. Vein of Galen), and aneurysms. Unfortunately, conventional approaches to guidewire and catheter delivery suffer from the shortcoming that substantial time may be required in selecting, shaping, and delivering the catheter intravenously to the surgical site. Time required for tool selection and vascular access increases the duration of patient anesthesia, infection risk, and corresponding costs based on operating room occupancy. Accordingly, configurations herein substantially improve the time for endoscopic procedures by selecting a guidewire and catheter based on a shape adapted to navigate the vascular pathway to the surgical site. A database or model of previous procedures and corresponding guidewires, catheters, and percutaneous access sites allows a comparison of patient anatomy and procedure to similar procedures and tools used in the previous procedures. Selection of an optimal guidewire shape, catheter, and percutaneous access site, along with a vascular pathway obtained from patient scans, expedites tool selection time and increases the likelihood of success with the selected guidewire, mitigating delays caused by successive insertion attempts.

In further detail, configurations discussed below present a method of selecting a surgical tool for an endovascular procedure by receiving an image of a surgical region including a surgical target, and identifying, based on a vascular path defined by the image, a surgical route to the surgical target. A server or app compares the vascular path to a database of previous procedures including catheters and guidewires, and identifies, based on the comparison of the vascular path to a corresponding previous procedure, a selected catheter and a selected guidewire based on the catheter and guidewire of the corresponding previous procedure. The database or model is generated from a plurality of previous procedures, where each of the previous procedures includes at least one catheter, and a guidewire having a shape defined by at least one bend having an angle.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of an endovascular surgical environment suitable for use with configurations herein;

FIGS. 2A-2B show human vasculature routes for surgical access in the environment of FIG. 1;

FIGS. 3A-3D show forces acting on vascular (blood vessel) structures and endoscopic instruments therein;

FIGS. 4A-4D show progression of a surgical catheter and guidewire through a vascular structure as in FIGS. 2A-2B;

FIG. 5 shows a classification of a vessel in anticipation of a traversal of the vascular path during a surgical procedure in the environment of FIG. 1;

FIGS. 6A-6D show shapes of vessels with candidate traversal paths; and

FIGS. 7A-7D show the respective wire shapes for traversal of the vessels of FIGS. 6A-6D.

### DETAILED DESCRIPTION

Endovascular surgical procedures such as neuroendovascular procedures are beneficial for treatment of ailments such as arteriovenous malformations (AVMs), cerebral aneurysms and ischemic strokes through Mechanical Thrombectomy (MT), often at considerable expense due to the time and expertise required.

The disclosed approach may also be employed with endoscopic approaches, meaning a scope with camera feedback, or endoluminal, referring to both endovascular and endoscopic capabilities, as in a surgery within an anatomical lumen. The claimed approach is equally suited to other forms of minimally invasive procedures using a flexible guided probe through a probe incision, in contrast to open surgery with an elogated lineal opening to a surgical field.

Conventional approaches typically employ a visual inspection of images of the patient's vessels to enable the surgeon to select an appropriate combination of guidewires, catheters, and percutaneous access site (femoral or radial) to reach diseased areas. The success of this process as well as its timely completion is very dependent on the experience of the surgeon. Vessels that are intricate and hard to navigate can involve substantial time in selecting combinations of tools, increasing procedural time and consequently: brain loss (for stroke procedures); radiation exposure for both patient and clinical personnel; injected contrast which may lead to renal failure; and costs associated with Operating Room (OR) time, anesthesia, and personnel effort.

An image analysis and selection application assists surgeons in making reliable strategic decisions on ideal tool usage and access sites, based on images of a patient's anatomy. The application processes preoperative images and compares them with a database of previous procedures to select the appropriate tools and puncture site based on the past procedures (historical intelligence). Alternate configurations may autonomously perform or simulate the procedure on a digital twin of the specific patient's anatomy (physical intelligence) based on the patient images, and finally provide the neuroendovascular surgeon with indications of optimal strategies derived from the matching or correspondence with the previous procedures.

Endovascular refers to minimally invasive, catheter-based procedures performed inside blood vessels (arteries or veins) to treat vascular diseases, rather than using traditional open surgery. It uses small, targeted incisions, typically radial or femoral, to insert catheters, stents, or balloons to repair vessels or remove blockages.

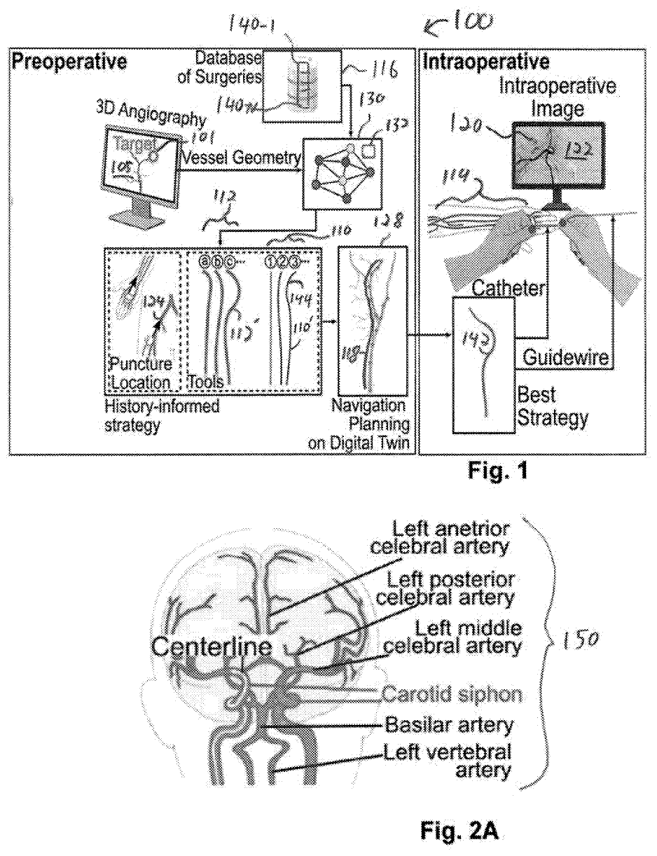

FIG. 1 is a context diagram of an endovascular surgical environment 100 suitable for use with configurations herein. Referring to FIG. 1, a general neuroendovascular procedure gathers preoperative imaging modalities such as Magnetic Resonance Imaging (MRI) and/or Computed Tomography (CT) to evaluate the nature of the disease. 3D images of the vessels such as Computed Tomography Angiography (CTA), and Magnetic Resonance Angiography (MRA) are used to locate a treatment site or target 101, and plan a navigation strategy. Tools such as guidewires and catheters are threaded through the vascular network 114 to reach the target 101 location in the anatomy, from an incision in the arm or leg of the patient (arterial puncture). During the intervention, fluoroscopic imaging or other suitable intraoperative imaging 120 provides concurrent feedback of catheter 112/guidewire 110 advancement and position. Typically contrast agents are employed to enhance visualization of the imaged vasculature 122. Once the target site is reached, treatment is delivered based on the disease, e.g. clot retrieval, embolization or aneurysms, to name several.

In addition to disease-specific complexities, neuroendovascular procedures generally share a common challenge of planning the navigation to the target location. The cerebral vasculature is highly intricate and patient-specific, requiring precise tool selection, accurate identification of the access point, and careful navigation to avoid complications. Neuroendovascular surgeons strive to select the appropriate combination of tools, i.e., guidewires, catheters, as well as the site for percutaneous access. Even the most experienced surgeons struggle with time constraints, image interpretation and complex tools handling.

The configuration of FIG. 1 addresses these shortcomings by a method of selecting a surgical tool for an endovascular procedure including receiving an image 105 of a surgical region including the surgical target 101, and identifying, based on a vascular path defined by the image, a surgical route to the surgical target 101. Based on the imaged vessel geometry, a server 130 invokes an application 132 for comparing the vascular path to a database 116 of previous procedures 140-1. . . 140-N (140 generally) including catheters 112 and guidewires 110. The application 132 identifies, based on the comparison of a vascular path 118 to a corresponding previous procedure 140′, a selected catheter 112′ and a selected guidewire 110′ with reference to the catheter and guidewire of the corresponding previous procedure 140.′ The selected tools 142 (guidewire 110′ and one or more catheters 112′) are rendered for physical invocation, and guided through the vascular network 114 facilitated by imaged guidance from the imaging 120.

A predicate step includes generating the database 116 from the plurality of previous procedures 140, such that each previous procedure of the plurality of previous procedures includes at least one catheter 112, and a guidewire 110, where the guidewire has a shape defined by at least one bend 144 having an angle and a length, typically expressed as a series of angled bends 144 running at a length to a successive bend.

In configurations herein, the comparison of the imaged vascular path 118 is based on an AI assistant leveraging historical intelligence from the imaged procedures and physical intelligence of the corresponding tool shape which can help surgeons of different experience levels make reliable and timely selections. The database 116 stores the previous procedures 140 of successful and less optimal past procedures, and may include a digital representation or “digital twin” 128 to refine the prediction, by planning the navigation of selected tools 142 advanced inside the digital twin 128. Reduction of tool selection time coupled with a high confidence from a simulated digital twin traversal of the vascular path 118 boosts the success rate. This involves generating the digital twin 128 based on the imaged vasculature 122 of the surgical region, and navigating the digital twin based on the shape of the selected/computed guidewire or tool 142 (tool collectively includes a guidewire and one or more catheter, depending on the procedure).

A historical or training corpus is employed for generating the database 116 from an aggregation of patient images resulting from scans of vascular structures traversed during the previous procedures 140. Each of the previous procedures 140 is associated with a guidewire 110 and at least one catheter 112, such that the guidewire has a shape based on the sequence of bends and segments to successive bends.

In operation in the environment of FIG. 1, the application 132 compares the vascular path 118 of the current procedure to the vascular structure of each of the previous procedures 140-1. . . 140-N. The application 132 computes a closest match to the previous procedures based on the comparison, and renders an indication of the selected guidewire and/or catheter tools 142 and the corresponding shape based on the guidewire associated with the closest match. The comparison may further include computing a set of current procedure features based on the vascular path 118, such that the set of current procedure features includes one or more angular bends, each angular bend including distance, a direction and an angle. Using the features computed for the current procedure, the application 132 classifies the set of current procedure features based on the database 116 or model for computing the selected catheter 110′ and a selected guidewire 112′. A match may be concluded by a high correspondence or probability of success based on a model of the features, or simply by a matching of numerical values for the bend angle, distance and direction, discussed further below.

The environment 100 of FIG. 1 depicts system for selecting a surgical tool for an endovascular procedure using an imaging interface to the imaging 120 medium configured to receive an image 122 of a surgical region including the surgical target 101. Using the interface to the imaging 120 medium and the database 116, the application 132 identifies, based on the comparison of the vascular path 118 to a corresponding previous procedure 140, the selected catheter 112′ and the selected guidewire 110′ based on the catheter and guidewire of the corresponding previous procedure 140.

A further configuration or enhancement renders the shape of the selected guidewire 110′, and then forms a patient specific guidewire based on the shape by advancing an unformed (straight) guidewire through a robotic claw or gripper for bending and advancing the guidewire based on the angular bend, run distance and angle of rotation of the bend.

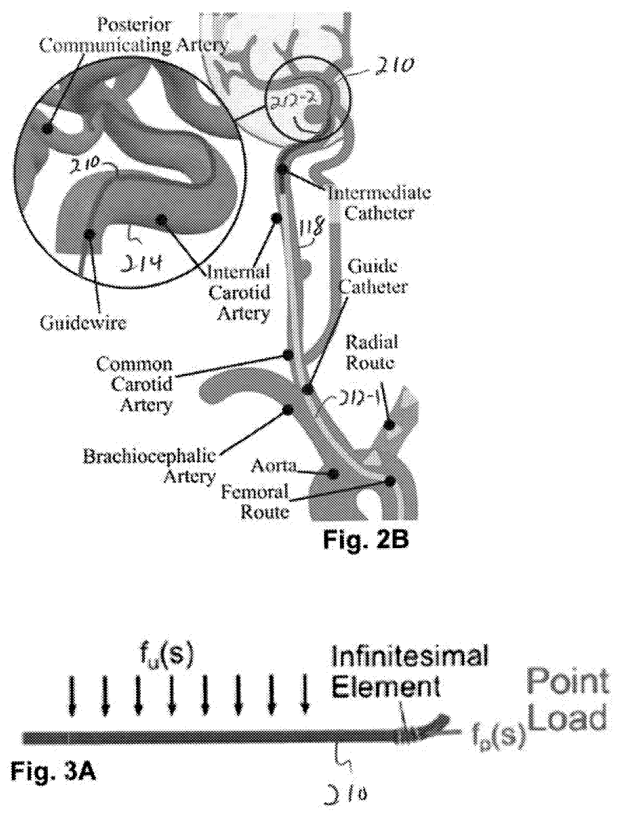

FIGS. 2A-2B show human vasculature routes for surgical access in the environment of FIG. 1. Referring to FIGS. 1 and 2A-2B, neuroendovascular procedures, such as mechanical thrombectomy for ischemic stroke, aneurysm embolization, and targeted embolotherapy, are complex interventions that require navigating tortuous (twisted, winding, or crooked, defined mathematically as the ratio of the actual curve length of a path to the straight-line distance between its endpoints) vasculature while operating under strict time constraints. These procedures are performed via radial or femoral access after creating an endovascular access path through which tools 142 are deployed from the imaged vasculature 122. The application 132 identifies the surgical route based on the vascular path from a percutaneous access site 124 to the surgical target 101.

Database 116 or model generation collects vessel images from a large corpus of patients. For each previous procedures 140, the database 116 accumulates 3D images of the brain vasculature 150, tools combinations which allowed successful navigation past the carotid siphon, and (if any) tools that did not lead to successful navigation.

By capturing the complexity of the vasculature, the database 132 assists in predicting whether femoral or radial access would be most successful. This involves measuring parameters such as path length, diameter and tortuosity. Similarly, will select the best combination of telescoping guidewire and catheter based on the most successful strategies in “similar” anatomies. To measure similarity, we will compare the centerline of the vessel to the ones in the database of collected anatomies.

FIG. 2B depicts catheterization for neuroendovascular treatments with alternative access routes via arm (radial) or leg (femoral). In FIG. 2B, the guidewire 210 navigates past the Internal Carotid Artery (ICA) into the Posterior Communicating Artery (PComm).

The clinical standard is to use a set of up to four coaxial tools, from distal to proximal: guide wire 210, microcatheter, intermediate catheter 212-2, and guide catheter 212-1 (212 generally). These passive tools are advanced through the vasculature and navigated under biplane fluoroscopy. Navigation leverages the natural shape of the tools and intentional contact with the vessel walls, while accounting for millimeter-scale vessel clearances, severe tortuosity, and dynamic vessel motion, all while managing radiation and contrast exposure. Although the guidewire 210 is designated as the tool 142 actually engaging the target 101, multiple catheters 112, 212 may also be employed and shaped.

The main element that drives navigation to the target location is the guidewire 110′, which is advanced to the target 101 and allows the microcatheter to track along it. The microcatheter is then often used to deliver treatment, e.g., in aneurysm treatments, or to further advance the intermediate catheter for aspiration in mechanical thrombectomy.

In routine conventional approaches, operators select pre-shaped wire geometries or manually shape wire and microcatheter tips to match an anticipated three-dimensional vascular geometry, often tortuous, branching, and highly variable across patients, and manually shape the wire tip with mandrels on the back table. Shaping neuroendovascular guidewires to match patient-specific anatomy to achieve optimal navigation and stability is a complex and highly skill-dependent process. Small deviations in curvature or orientation can result in failure to access the target vessel, excessive contact with the vessel wall, or loss of support for subsequent catheter advancement. This process typically relies on the surgeon's experience and visual estimation from preoperative or fluoroscopic images rather than quantitative feedback which makes reproducibility difficult. Furthermore, the guidewire 110′ typically maintains a shape once inserted, but still needs to be flexible enough to follow the vessel path, requiring a delicate balance of stiffness and elasticity that is challenging to achieve consistently. Stiffness and/or flexibility may be a further parameter or feature associated with the previous procedures 140.

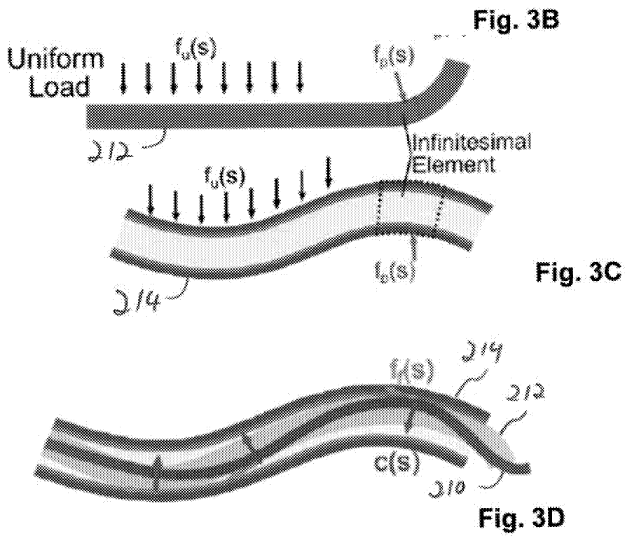

FIGS. 3A-3D show forces acting on vascular (blood vessel) structures and endoscopic instruments therein. Referring to FIGS. 1-3D, navigation through the vascular path 118 is planned using the database 116 or equivalent AI model in a digital twin of the patient's anatomy based on the patient imaging. The model is based on the simplified assumption that guidewire 210, catheter 212, and vessels 214 can be considered as independent tubular elements of specific physical characteristics such as stiffness and friction and respond to external forces along their length (fᵤ, fₚ).

The application 132 then applies constraints that the portion of the guidewire 210 lies within the catheter 212, and both are contained within the vessel 214 which leads to constraining forces (c(s)) and friction (f(s)) to arise between the elements.

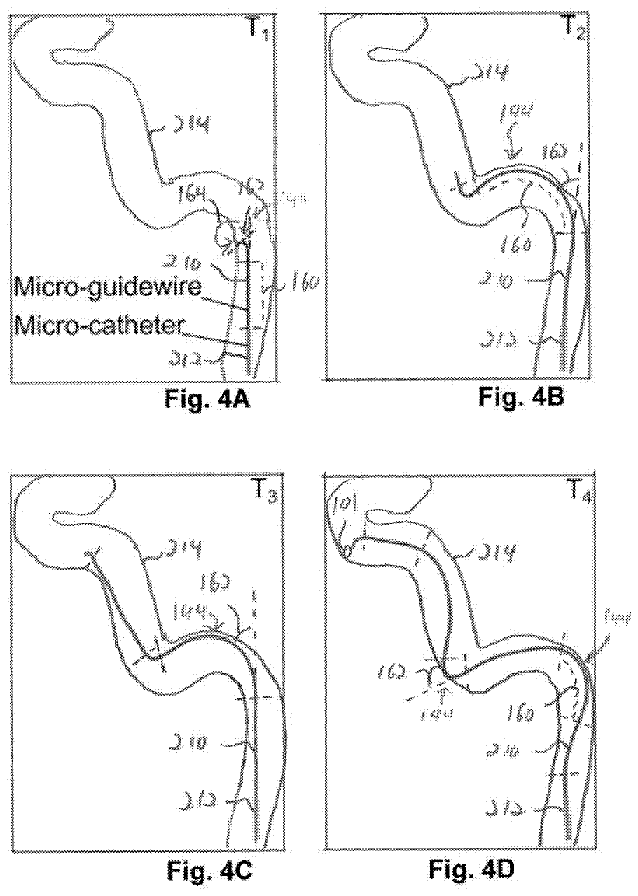

FIGS. 4A-4D show progression of a surgical catheter and guidewire through a vascular structure as in FIGS. 2A-2B. Referring to FIGS. 1-4D, FIGS. 4A-4D show that the shape of the guidewire 210 further comprises a series of angular bends 144 each having a distance 160, a direction 164 and an angle 162. The sequence of bends is invoked for identifying an access route through the patient vascular 214 structure to the surgical target 101 based on the image, such that the selected guidewire 110′ and catheter 112′ have a shape for navigating the access route. Note that a straight run can be defined as the trivial case of a zero angle and zero direction with an appropriate length or distance.

FIG. 5 shows a classification of a vessel in anticipation of a traversal of the vascular path during a surgical procedure in the environment of FIG. 1. FIG. 5 depicts intraoperative imaging 120 in three orientations, 120-1 . . . 120-3 imaging the selected guidewire 110′ traversing the vessel 214 as tracked via the imaging 120-1 . . . 120-3. In a particular configuration, the database 116 of previous procedures 140 includes a model based on features including, for each previous procedure 140, an indication of at least one catheter 212, and a guidewire 210 including one or more angular bends, each angular bend including a distance 160, a direction 164 and an angle 162. As shown by the figures, the angle 162 refers to a degree of curvature from a central axis, and the direction refers to a rotary or polar direction outward from the central axis, as in a rotation around the central axis through the guidewire.

The application 132 is suitable for execution on the server 130 using any suitable operating system and user interface for launching the application, and the database 116 is in communication with the application 116 via a connection with the server 130.

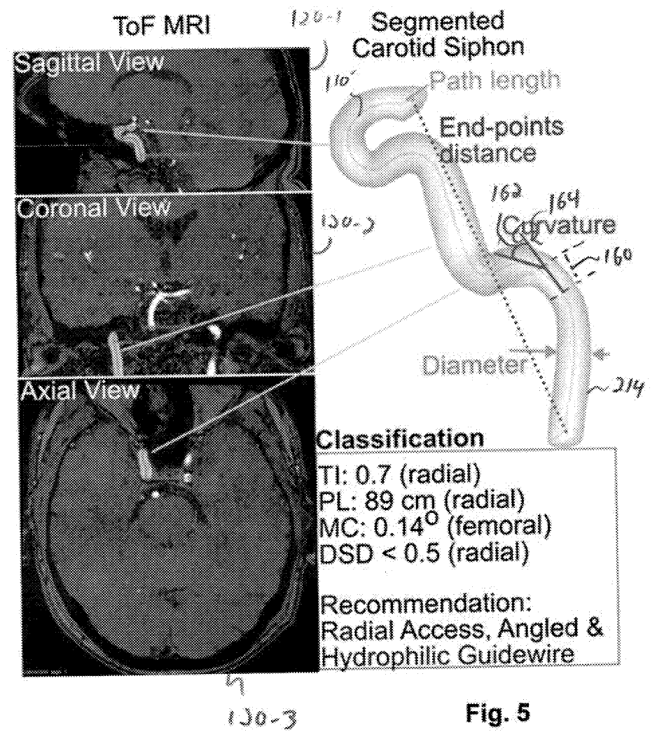

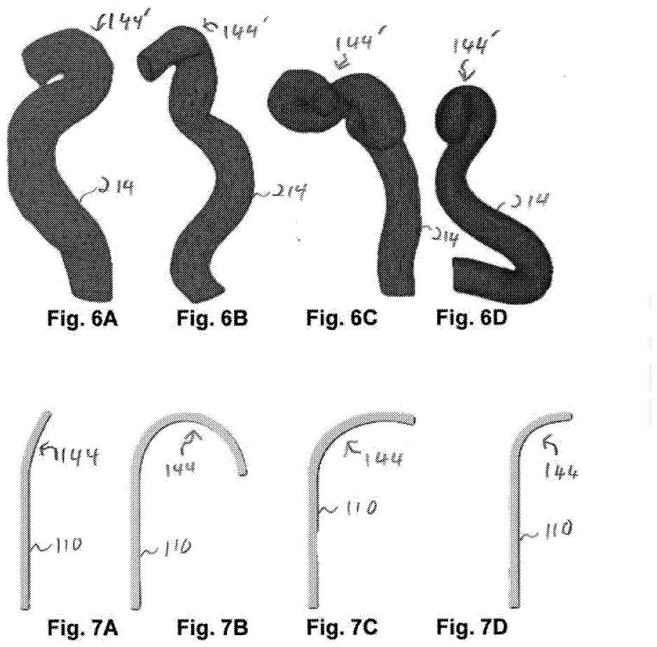

FIGS. 6A-6D show shapes of vessels 214 with candidate traversal paths, and FIGS. 7A-7D show the respective wire shapes for traversal. For the vessels 214 in FIGS. 6A-6D, each wire 110 has a bend 144 shaped to correspond to a curve 144′ in the respective vessel 214.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of selecting a surgical tool for an endovascular procedure, comprising:
receiving an image of a surgical region including a surgical target;
identifying, based on a vascular path defined by the image, a surgical route to the surgical target;
comparing the vascular path to a database of previous procedures including catheters and guidewires; and
identifying, based on the comparison of the vascular path to a corresponding previous procedure, a selected catheter and a selected guidewire based on the catheter and guidewire of the corresponding previous procedure.

2. The method of claim 1 further comprising generating the database from a plurality of previous procedures, each previous procedure of the plurality of previous procedures including:
at least one catheter; and
a guidewire, the guidewire having a shape, the shape defined by at least one bend having an angle.

3. The method of claim 2 wherein the shape further comprises a series of angular bends each having a distance, a direction and an angle.

4. The method of claim 1 further comprising identifying the surgical route based on a vascular path from a percutaneous access site to the surgical target.

5. The method of claim 1 further comprising:
generating the database from an aggregation of patient images resulting from scans of vascular structures traversed during the previous procedures; and
associating each of the previous procedures with a guidewire and at least one catheter, the guidewire having a shape.

6. The method of claim 5 further comprising:
comparing the vascular path to the vascular structure of each of the previous procedures;
computing a closest match to the previous procedures based on the comparison; and
rendering the selected guidewire and the corresponding shape based on the guidewire associated with the closest match.

7. The method of claim 1 further comprising identifying an access route through the patient vascular structure to the surgical target based on the image, the selected catheter having a shape for navigating the access route.

8. The method of claim 1 wherein the database of previous procedures includes a model based on features including, for each previous procedure, an indication of at least one catheter, and a guidewire including one or more angular bends, each angular bend including distance, a direction and an angle.

9. The method of claim 8 wherein the comparison further comprises:
computing a set of current procedure features based on the vascular path, the set of current procedure features including one or more angular bends, each angular bend including distance, a direction and an angle; and
classifying the set of current procedure features based on the model for computing the selected catheter and a selected guidewire.

10. The method of claim 1 further comprising forming a patient specific guidewire based on the shape.

11. The method of claim 2 further comprising:
generating a digital twin based on the image of the surgical region; and
navigating the digital twin based on the shape.

12. A system for selecting a surgical tool for an endovascular procedure
an imaging interface to an imaging medium, the imaging interface configured to receive an image of a surgical region including a surgical target;
a database for identifying, based on a vascular path defined by the image, a surgical route to the surgical target;
an application configured to
compare the vascular path to a database of previous procedures including catheters and guidewires, and
identify, based on the comparison of the vascular path to a corresponding previous procedure, a selected catheter and a selected guidewire based on the catheter and guidewire of the corresponding previous procedure.

13. The system of claim 12, wherein the database is generated from a plurality of previous procedures, each previous procedure of the plurality of previous procedures including:
at least one catheter; and
a guidewire, the guidewire having a shape, the shape defined by at least one bend having an angle.

14. The system of claim 13 wherein the shape further comprises a series of angular bends each having a distance, a direction and an angle.

15. A computer program embodying program code on a non-transitory computer readable storage medium that, when executed by a processor, performs steps for implementing a method of selecting a surgical tool for an endovascular procedure, the method comprising:
receiving an image of a surgical region including a surgical target;
identifying, based on a vascular path defined by the image, a surgical route to the surgical target;
comparing the vascular path to a database of previous procedures including catheters and guidewires; and
identifying, based on the comparison of the vascular path to a corresponding previous procedure, a selected catheter and a selected guidewire based on the catheter and guidewire of the corresponding previous procedure.
